(19) United States
(12) Patent Application Publication
(10) Pub. No.: US 2007/0172525 A1
(43) Pub. Date: Jul. 26, 2007

(54) Anti-diabetic combinations

(76) Inventor: Ramesh Sesha, West Windsor, NJ (US)

Correspondence Address: Ramesh Sesha, 9113 Taylor Court, West Windsor, NJ, 08550, US

(21) Appl. No.: 11/724,486

(22) Filed: Mar. 15, 2007

(51) Int. Cl. A61K 31/41; A61K 31/155; A61K 9/22

(52) U.S. Cl. 424/468; 514/635; 514/381

(57) ABSTRACT

This invention a pharmaceutical composition comprising a DPP inhibitor and a slow release biguanide. The invention further discloses a method of administering a combination comprising a DPP inhibitor and a slow release biguanide to a mammal in need of thereof.

This invention describes a pharmaceutical composition comprising a DPP inhibitor and a slow release biguanide. The invention further discloses a method of administering a combination comprising a DPP inhibitor and a slow release biguanide to a mammal in need of thereof.

### BACKGROUND OF THE INVENTION

Diabetes mellitus of type II is a progressive metabolic disorder with diverse pathologic manifestations and is often associated with lipid metabolism and glycometabolic disorders. The long-term effects of diabetes result from its vascular complications; the microvascular complications of retinopathy, neuropathy and nephropathy and the macrovascular complications of cardiovascular, cerebrovascular and peripheral vascular diseases. Initially, diet and exercise is the mainstay of treatment of type II diabetes. However, these are followed by administration of oral hypoglycemic agents. Current drugs used for managing type II diabetes and its precursor syndromes such as insulin resistance include classes of compounds, such as, among others, biguanides, glitazones and sulfonylureas¹.

Dipeptidyl peptidase (DPP4) inhibitors, that include Sitagliptin, Vildagliptin and Saxagliptin, are a new class of drugs that inhibit the proteolytic activity of dipeptidyl peptidase-4, thereby potentiating the action of endogenous glucoregulatory peptides, known as incretins. They are orally-bioavailable selective DPP4 inhibitors that were discovered through the optimization of a class of -amino-acid-derived DPP4 inhibitors. It lowers DPP4 activity in a sustained manner following once daily administration, preserves the circulating levels of intact GIP and GLP1 following meals in both acute and chronic studies and reduces blood glucose levels without significant increases in hypoglycaemia².

Glitazones, represented principally by the class of glitazones including, for example, rosiglitazone, troglitazone and pioglitazone, among others, act by increasing the sensitivity of insulin receptors in the body and decreasing peripheral insulin resistance. Glitazones, preferably pioglitazone, stimulate adipogenesis and reduce plasma triglyceride and free fatty acid concentrations. These enhance insulin action at the cellular level but do not stimulate insulin release, nor do they mimic its action^{1,3}

Sulfonylureas, represented principally by glipizide, glimiperide, glyburide, glibornuride, glisoxepide, gliclazide acetohexamide, chlorpropamide, tolazamide, and tolbutamide, among others, help in controlling or managing NIDDM by stimulating the release of endogenous insulin from the beta cells of the pancreas^{1 3}.

Biguanides represented principally by metformin, phenformin and buformin, help in the control of blood glucose by inhibiting hepatic glucose production, reducing intestinal absorption of glucose and enhancing peripheral glucose uptake. Biguanides, especially metformin, lowers both basal and post-prandial plasma glucose and thus improves tolerance of glucose in patients. Metformin exerts normoglycemic action with reduced risk of lactic acidosis and is also known to lower blood triglyceride levels. It is therefore a preferred mode of therapy among biguanides. Metformin is widely viewed as the initial drug of choice for the treatment of T2DM, owing to its 30-year track record, efficacy, safety and low cost. However, many physicians now advocate initiating therapy of T2DM with at least two drugs to obviate the monotherapy failure that accompanies prolonged metformin use in the majority of treated patients^{1 3 & 4}

DPP4 inhibitors, biguanides, glitazones and sulfonylureas are commercially available in the form of tablets of the individual drugs, either as immediate release (IR) formulations or in some cases controlled release (CR) formulations, to be administered orally to patients in need thereof, in protocols calling for the single administration of the individual ingredient. Metformin monotherapy is used as a first line treatment in diabetic patients but may be supplemented with other drugs when the secondary failure of the therapy sets in. The addition of a DPP inhibitor, glitazones and sulfonylurea to the concurrent treatment provides a balance of stimulated release of insulin while ameliorating insulin resistance and thus provides an optimal level of glycemic control unattainable by either medication alone. But, multiple medications such as these for the prophylaxis or treatment of diseases usually result in patient inconvenience and consequently, patient non-compliance to the prescribed dosage regimen. The ease of using combination therapy for multiple medications as opposed to separate administrations of the individual medications has long been recognized in the practice of medicine. Such a therapy provides therapeutic advantage for the benefit of the patient and the clinician. Further, such therapy provides both increased convenience and improved patient compliance resulting form the avoidance of missed doses through patient forgetfulness.

A brief logical profile for such combinations based on the pharmacological mechanism of action of the individual classes of drugs is given below:

Insulin resistance and reduced insulin secretion are the two fundamental abnormalities in type 2 diabetic patients. Therefore, reducing insulin resistance or increasing insulin sensitivity and augmenting insulin secretion from beta cells of pancreas are the two major treatment approaches. The tissues most commonly resistant to actions of insulin are liver, skeletal muscles, and adipose tissues. Therefore, treatment strategies directed towards improving the insulin sensitivity of these major tissues help in overall enhancement of insulin sensitivity.

It is known that Pioglitazone plays a major role in improving sensitivity of peripheral tissues like skeletal muscles and adipose tissues whereas Metformin has its primary action on liver. Therefore, the combination therapy with Pioglitazone or Rosiglitazone and Metformin results in synergistic actions to improve insulin sensitivity.

Pioglitazone, a member of the thiazolidinedione class of anti-diabetic agents, targets insulin resistance by binding to the transcription factor peroxisome proliferators activated receptors (PPAR-γ), promoting synthesis of glucose transporters. It enhances insulin sensitivity, thereby reducing hyperglycemia, glycosylated haemoglobin (HbA1c), hyperinsulinemia and hypertriglyceridemia.

In contrast, Metformin hydrochloride promotes glucose lowering by reducing hepatic glucose production and gluconeogenesis and by enhancing peripheral glucose uptake. Because Metformin and Pioglitazone act through different mechanisms, their combined use is indicated in patients whose disease is poorly controlled with monotherapy.

The safety and efficacy of a DPP inhibitor, for example sitagliptin as a monotherapy and in combination with existing anti-diabetic agents was assessed in four randomized double-blind placebo-controlled clinical trials that involved more than 2,000 patients with T2DM^{6, 7, 9, 10}, Several measurements relevant to glycemic control were evaluated, including the mean change from baseline in glycated hemoglobin (HbA1C) levels—an indicator of average blood-sugar levels for the past 3-4 months. Sitagliptin as a monotherapy at doses of either 100 or 200 mg daily significantly reduced HbA1C, with few adverse events, and no significant increase in hypoglycemia^{7, 8}. The extent of HbA1C reduction was proportional to the starting HbA1C, and no significant weight gain was observed in 24-week monotherapy studies. Sitagliptin reduced both fasting and postprandial glycaemia, in association with improvements in the proinsulin/insulin ratio and homeostatic model assessment of −cell function (HOMA-B)⁸. For patients who did not achieve adequate glycemic control on at least 1,500 mg per day of metformin (mean HbA1C of 8%), the addition of sitagliptin 100 mg daily resulted in 47% of patients achieving a HbA1C of <7%, compared with 18.3% of placebo-treated subjects⁹. The mean placebo-subtracted reduction in HbA1C was 0.65%, and sitagliptin therapy was also associated with significant reductions in fasting glucose and increases in parameters of −cell function. Sitagliptin has also been shown to be effective when combined with metformin as initial therapy for T2DM. In 24-week studies of sitagliptin as an add-on therapy for patients not achieving adequate glycemic control (mean HbA1C 8.1%) on pioglitazone (30 or 45 mg daily), sitagliptin at a dose of 100 mg daily produced a mean HbA1C reduction of 0.7%, and significantly greater numbers of patients achieved a HbA1C of <7% on sitagliptin relative to pioglitazone alone (45.4 versus 23%, respectively)¹⁰. Sitagliptin therapy was not associated with increased rates of hypoglycemia or weight gain relative to patients treated with pioglitazone alone.

Metformin SL is a modified release gastro-retentive formulation⁵ and the slow release is achieved using a number of different technologies (U.S. Pat. Nos. 6,099,859, 6,340,475, 6,403,121, 6,475,521, 6,676,966) By virtue of its gastro-retentive property, a slow release delivery system releases Metformin gradually in small amounts, which is well absorbed in the upper part of the small intestine and duodenum. Metformin incorporated into the gastro-retentive formulation is released slowly over a prolonged period of 24 hours; hence given once a day. Metformin SL has distinct advantages over plain Metformin which are as follows:
1. It reduces the number of daily doses and increases patient compliance. As treatment of diabetes is life-long, this aspect is very important from a patient's point of view.
2. Metformin SL, being a modified release preparation can also avoid “dose-loading”. This commonly occurs with conventional oral formulations when large doses are given which may cause sudden release and absorption of a large amount of drug.
3. Metformin SL is released in smaller doses in upper part of the small intestine, and hence ensures increased bioavailability and decreased side effects. In contrast, conventional Metformin has lesser bioavailability since its absorption decreases as it passes through the lower part of small intestine.
4. Conventional Metformin has an, oral bioavailability of 40 to 60% and gastrointestinal absorption is apparently complete within 6 hours of ingestion. Plasma t ½ is 2 to 6 hours. Hence it has to be given 2 to 3 times a day, whereas Metformin SL being a controlled release “gastro-retentive” formulation, is released in small quantities in upper part of small intestine where the drug is better absorbed and has a prolonged duration of action (24 hours).
5. Metformin SL—the absorption is more dependable and complete as the drug is released gradually mainly in the upper part of small intestine, whereas in Metformin plain the absorption is erratic as Metformin is also absorbed in the latter part of small intestine where absorption is erratic and “non-dependable”.
6. Since Metformin SL is released slowly, side effects like flatulence, abdominal discomfort, diarrhea and lactic acidosis are less unlike plain Metformin.
7. An inverse relationship was observed between the dose ingested and relative absorption with therapeutic doses ranging from 0.5 to 1.5 gm suggesting the involvement of an active, saturable absorption process. Thus a slow release formulation of Metformin can not only optimizes the daily requirement of Metformin, but can also reduce the need of a higher dose.

Pharmaceutical dosage forms containing combinations of anti-diabetic drugs have been proposed in the art. For example, EPO 0 749 751 (which is incorporated herein by reference) teaches pharmaceutical compositions comprising an insulin sensitivity enhancer, which could be a thiazolidinedione compound, in combination with other anti-diabetics. More specifically, EPO 0 749 751 teaches that the preferred insulin sensitivity enhancer is pioglitazone, which can be combined with other anti-diabetics such as metformin, phenformin or buformin, and further that these drugs can be associated (mixed and/or coated) with conventional excipients to provide taste masking or sustained release behavior. Another example of a combination of antihyperglycemic drugs and thiazolidinedione derivatives is U.S. Pat. No. 6,011,049, which is incorporated herein by reference. This patent teaches a single pharmaceutical composition that contains pioglitazone or trolitazone and metformin in slow release forms such as osmotic pumps or skin patches. Other combinations of antihyperglycemic drugs and thiazolidinedione derivatives can be found in U.S. Pat. Nos. 6,524,621; 6,475,521; 6,451,342 and 6,153,632 and PCT patent applications WO 01/3594 and WO 01/3594, which are incorporated herein by reference. U.S. Pat. No. 7,125,873 describes pharmaceutical composition comprising a DPP4 inhibitor like Sitagliptin with other anti-diabetic drugs like biguanide, PPAR agonists

Although the prior art teaches pharmaceutical dosage formulations that contain combination drugs, the present invention provides numerous benefits over the prior art teaching. It is an object of the present invention to provide a pharmaceutical composition comprising a DPP4 inhibitor and a slow release biguanide. Further it is also an object of the present invention to provide a method of administering the combination of a slow release biguanide and a DPP4 inhibitor that provide the following advantages
1. The combination targets the two major pathological processes, insulin resistance, and potentiation of glucose-dependent insulin secretion using a combination of slow release biguanide and a DPP4 inhibitor.
2. The therapeutic objective is achieved with the combination of a slow release Biguanide and DPP4 inhibitor irrespective what biguanide formulation is used to affect its slow release
3. Increased insulin sensitivity due to synergistic actions of DPP4 inhibitor and a slow release biguanide
4. Therapeutic actions of Metformin are enhanced due to its slow release over a period of time.
5. Better glycemic control because of using a slow release and an immediate release
6. Reduced incidence of side effects due reduced dosage requirements of individual drugs.
7. Once a day administration
8. Improved compliance

It is an object of the present invention to provide a pharmaceutical dosage comprising a DPP4 inhibitor and a slow release biguanide

It is further an object of the present invention to provide a method of administering a pharmaceutical composition comprising a DPP4 inhibitor and a slow release biguanide.

It is another object of the present invention to provide a pharmaceutical kit comprising a DPP4 inhibitor and a slow release biguanide

It is an additional object of the present invention to provide a dosage form comprising delivery of a DPP inhibitor and a biguanide wherein the peak plasma levels of the biguanide compound is approximately 8-12 hours after administration and peak plasma levels of a DPP inhibitor is approximately 1-4 hours after dosing.

It is yet another object of the present invention to provide a pharmaceutical composition as described above, comprising delivery of a biguanide as a slow release formulation in combination with delivery of a second active drug by immediate release comprising a DPP4 inhibitor that can provide continuous and non-pulsating therapeutic levels of said biguanide to an animal or human in need of such treatment over a eight hour to twenty-four hour period.

Further it an object of the present invention to provide a pharmaceutical composition comprising a biguanide as a controlled or sustained release component and a DPP4 inhibitor as a immediate release component, wherein not less than 85% of the total amount of the DPP 4 inhibitor is released from the dosage form within 120 minutes or less.

### SUMMARY OF THE INVENTION

It is therefore an object of the invention is to provide efficacious methods for the development of drug delivery systems of combination of a slow release Metformin and a DPP4 inhibitor. Furthermore, in light of the foregoing, the principal object of the present invention is to provide a delivery system for oral administration of a combination of slow release drug and a DPP4 inhibitor. A typical example for such a combination providing glycemic control to diabetic patients include a sustained/controlled/extended release biguanide in combination with a an immediate release drugs such DPP4 inhibitor.

It is another object of the present invention to provide a method of administrating a combination comprising a slow release biguanide and a DPP4 inhibitor that release in the body of a mammal, a sustained release biguanide and a DPP inhibitor.

It is yet another object of the present invention is to provide an oral delivery system kit which comprises of a slow release biguanide and DPP4 inhibitor wherein the biguanide is combined with a DPP4 inhibitor, in anyway using any slow release drug delivery system.

These objects are achieved by virtue of the present invention, which provides an oral delivery system that selectively delivers drugs at an optimal rate to patients over a period of time during treatment and aims to achieve a reduction in the dose of drugs administered after an initial therapy with this regimen. The reduction in dosage shall be beneficial to the patient and will be at the discretion of the medical doctor depending upon the pathological profile obtained after treatment with this combination. Further this invention provides for method of administering any slow release biguanide with a DDP4 inhibitor in achieving the therapeutic objective.

### DETAILED DESCRIPTION OF THE INVENTION

The term, “biguanide” as used in this specification, refers to drugs that are useful in controlling or managing noninsulin-dependent diabetes mellitus (NIDDM). They include the biguanides such as metformin, phenformin or buformin or the like, and pharmaceutically acceptable salts, isomers or derivatives thereof.

The term “DPP4 Inhibitor” as used in this specification refers to drugs that are useful for controlling or managing NIDDM. These include, but are not limited to, Sitagliptin, Saxagliptin, Vildagliptin, other molecular entities such as SYR 522 (pyrimidine derivatives), PHX 1149, GRC-8200 (tricyclic derivatives), SSR162369 (biocyclic 8-pyrrolidinoxanthine) derivatives that inhibit DPP4 protease in a mammal²

The term “diabetes” as employed herein refers to Type 2 diabetes and Type 1 diabetes, usually Type 2 diabetes.

The term “slow-release” here applies to any release from a formulation that is other than an immediate release wherein the release of the active ingredient is slow in nature. This includes various terms used interchangeably in the pharmaceutical context like extended release, delayed release, controlled release, timed release, specific release, targeted release etc

The term “extended release material” as present in the inner solid particulate phase and the outer solid continuous phase refers to one or more hydrophilic polymers and/or one or more hydrophobic polymers and/or one or more other type hydrophobic materials, such as, for example, one or more waxes, fatty alcohols and/or fatty acid esters. The “extended release material” present in the inner solid particulate phase may be the same as or different from the “extended release material” present in the outer solid continuous phase.

The term “candidate for sustained release” encompasses all the characteristics of a drug which make it a candidate for formulating it into an extended release fashion like a short elimination half life and consequent dosing of more than once a day, a single dose product given in an extended fashion to achieve better clinical results and avoid side effects associated with an immediate release etc

The term “binding agent” as used in this specification, refers to any conventionally known pharmaceutically acceptable binder such as polyvinyl pyrrolidone, hydroxypropyl cellulose, hydroxyethyl cellulose, hydroxypropyl methylcellulose, ethylcellulose, polymethacrylate, polyvinylalcohol, waxes and the like. Mixtures of the aforementioned binding agents may also be used. The preferred binding agents are water soluble materials such as polyvinyl pyrrolidone having a weight average molecular weight of 25,000 to 3,000,000. The binding agent may comprise approximately about 0 to about 40% of the total weight of the core and preferably about 3% to about 15% of the total weight of the core. In one embodiment, the use of a binding agent in the core is optional.

The term “gelling or swelling polymer” as used in this specification, refers to polymers that gel, swell or expand in the presence of water or biological fluids. Representative examples of gelling or swelling polymers are high molecular weight hydroxpropyl methylcellulose (such as METHOCEL® K100M, which is commercially available from Dow Chemical) and high molecular weight polyethylene oxides (such as POLYOX WSR 301, WSR 303 or WSR COAGULANT). Other gelling or swelling polymers are described in U.S. Pat. No. 4,522,625 (which is incorporated herein by reference).

The term “seal coat’ as defined in this invention is a coating that does not contain an active pharmaceutical ingredient and that rapidly disperses or dissolves in water.

A pore forming is preferably a water-soluble material such as sodium chloride, potassium chloride, sucrose, sorbitol, mannitol, polyethylene glycols (PEG), propylene glycol, hydroxypropyl cellulose, hydroxypropyl methycellulose, hydroxypropyl methycellulose phthalate, cellulose acetate phthalate, polyvinyl alcohols, methacrylic acid copolymers, poloxamers (such as LUTROL F68, LUTROL F127, LUTROL F108 which are commercially available from BASF) and mixtures thereof.

The term “Hydrophilic polymers” as used in this specification include, but are not limited, to hydroxypropylmethylcellulose, hydroxypropylcellulose, sodium carboxymethylcellulose, carboxymethylcellulose calcium, ammonium alginate, sodium alginate, potassium alginate, calcium alginate, propylene glycol alginate, alginic acid, polyvinyl alcohol, povidone, carbomer, potassium pectate, potassium pectinate, etc

The term “Hydrophobic polymers” as used in this specification include, but are not limited, to ethyl cellulose, hydroxyethylcellulose, ammonio methacrylate copolymer (Eudragit RL™ or Eudragit RS™), methacrylic acid copolymers (Eudragit L™ or Eudragit S™), methacrylic acid-acrylic acid ethyl ester copolymer (Eudragit L 100-5™), methacrylic acid esters neutral copolymer (Eudragit NE 30D™), dimethylaminoethylmethacrylate-methacrylic acid esters copolymer (Eudragit E 100™), vinyl methyl ether/maleic anhydride copolymers, their salts and esters (Gantrez™) etc.

Other hydrophobic materials which may be employed in the inner solid particulate phase and/or outer solid continuous phase include, but are not limited, to waxes such as beeswax, carnauba wax, microcrystalline wax, and ozokerite; fatty alcohols such as cetostearyl alcohol, stearyl alcohol; cetyl alcohol myristyl alcohol etc; and fatty acid esters such as glyceryl monostearate, glycerol monooleate, acetylated monoglycerides, tristearin, tripalmitin, cetyl esters wax, glyceryl palmitostearate, glyceryl behenate, hydrogenated castor oil, etc.

The present invention concerns a pharmaceutical composition or dosage form comprising a slow release biguanide as the first active ingredient and a DPP4 inhibitor as the second active ingredient. Further, biguanide is preferably a metformin or a pharmaceutically acceptable salt thereof and is delivered in a controlled release manner from a tablet core, preferably an osmotic tablet core with or without a gelling or swelling polymer. The tablet core should include the biguanide and at least one pharmaceutically acceptable excipient. In one embodiment of the present invention the tablet core includes the biguanide, a binding agent and an absorption enhancer, and the tablet core is preferably coated with a polymeric coating to form a membrane around the tablet and drilled to create one passageway on each side of the membrane. The second active ingredient comprises a DPP4 inhibitor or its pharmaceutically equivalent salt, and is preferably applied to the membrane of the tablet core and provides for either immediate or controlled release of said DPP4 inhibitor.

In a preferred embodiment, the use of an absorption enhancer is optional and it can be any type of absorption enhancer commonly known in the art such as a fatty acid, a surfactant (anionic, cationic, amphoteric), a chelating agent, a bile salt or mixtures thereof. Examples of some preferred absorption enhancers are lecithin, fatty acids such as capric acid, oleic acid and their monoglycerides, surfactants such as sodium lauryl sulfate, sodium taurocholate and polysorbate 80, chelating agents such as citric acid, phytic acid, ethylenediamine tetraacetic acid (EDTA) and ethylene glycol-bis(.beta.-aminoethyl ether)-N,N,N,N-tetraacetic acid (EGTA). The core may comprise approximately 0 to about 20% of the absorption enhancer based on the total weight of the core and most preferably about 2% to about 10% of the total weight of the core.

In one embodiment of the present invention, the core of the present invention is preferably formed by granulating a biguanide with a binding agent and compressing the granules with the addition of a lubricant and absorption enhancer into a tablet and this embodiment doesn't use a gelling or swelling polymer. The core may also be formed either by dry granulating the core ingredients by passing them through a roller compactor and compressing the granules with the addition of a lubricant into tablets or by direct compression. It can also be achieved using other commonly known granulation procedures that are known in the art. This is only an example as, other excipients such as lubricants, pigments or dyes may also be employed in the formulation of the subject invention.

A membrane or sustained release coating is used as a coat in the core as outlined in this specification. Materials that are useful in forming the membrane or slow release coating are ethylcellulose, cellulose esters, cellulose diesters, cellulose triesters, cellulose ethers, cellulose ester-ether, cellulose acylate, cellulose diacylate, cellulose triacylate, cellulose acetate, cellulose diacetate, cellulose triacetate, cellulose acetate propionate and cellulose acetate butyrate. Other suitable polymers are described in U.S. Pat. Nos. 3,845,770; 3,916,899; 4,008,719; 4,036,228 and 4,612,008 which are incorporated herein by reference. Cellulose acetate, comprising an acetyl content of 39.3 to 40.3%, and is commercially available from Eastman Fine Chemicals, is the most preferred membrane or slow release coating

Further in an alternative embodiment, a flux-enhancing agent can also be included in the membrane or slow release coating can include one of the above-described polymers. The flux enhancing agent can increase the volume of fluid imbibed into the core to enable the dosage form to dispense substantially all of the biguanide through the passage and/or the porous membrane. The flux-enhancing agent can be a water-soluble material or an enteric material. Examples of the preferred materials that are useful as flux enhancers include but not limited to sodium chloride, potassium chloride, sucrose, sorbitol, mannitol, polyethylene glycols (PEG), propylene glycol, hydroxypropyl cellulose, hydroxypropyl methycellulose, hydroxypropyl methycellulose phthalate, cellulose acetate phthalate, polyvinyl alcohols, methacrylic acid copolymers, poloxamers (such as LUTROL F68, LUTROL F127, LUTROL F108 which are commercially available from BASF) and mixtures thereof. A preferred flux-enhancer used in this invention is PEG 400.

The flux enhancer may also be a water soluble drug such as metformin or its pharmaceutically acceptable salts, or the flux enhancer may be a drug that is soluble under intestinal conditions. If the flux enhancer is a drug, the present pharmaceutical composition has an added advantage of providing an immediate release of the drug that has been selected as the flux enhancer. The flux enhancing agent dissolves or leaches from the membrane or sustained release coating to form channels in the membrane or sustained release coating which enables fluid to enter the core and dissolve the active ingredient. In the preferred embodiment, the flux enhancing agent comprises approximately 0 to about 40% of the total weight of the coating, most preferably about 2% to about 20% of the total weight of the coating.

A commonly known excipient such as a plasticizer may also be used for preparing the membrane or slow release coating Some commonly known plasticizers include but not limited to adipate, azelate, enzoate, citrate, stearate, isoebucate, sebacate, triethyl citrate, tri-n-butyl citrate, acetyl tri-n-butyl citrate, citric acid esters, and all those described in the Encyclopedia of Polymer Science and Technology, Vol. 10 (1969), published by John Wiley & Sons. The preferred plasticizers are triacetin, acetylated monoglyceride, grape seed oil, olive oil, sesame oil, acetyltributylcitrate, acetyltriethylcitrate, glycerin sorbitol, diethyloxalate, diethylmalate, diethylfumarate, dibutylsuccinate, diethylmalonate, dioctylphthalate, dibutylsebacate, triethylcitrate, tributylcitrate, glyceroltributyrate and the like. Though the exact amount used depends on the type of plasticizer used, typically amounts from about 0 to about 25% are used, and preferably about 2% to about 15% of the plasticizer can be used based upon the total weight of the membrane or sustained release coating.

Generally, the membrane or slow release coating around the core will comprise from about 1% to about 10% and preferably about 2% to about 5% based upon the total weight of the core and coating.

The membrane or sustained release coating surrounding the core further comprises a passage that will allow for controlled release of the drug from the core in a preferred embodiment. As used herein the term passage includes an aperture, orifice, bore, hole, weakened area or an credible element such as a gelatin plug that erodes to form an osmotic passage for the release of the biguanide from the dosage form. Passage used in accordance with the subject invention are well known and are described in U.S. Pat. Nos. 3,845,770; 3,916,899; 4,034,758; 4,077,407; 4,783,337 and 5,071,607.

The present invention provides a combination that includes a DPP4 inhibitor that is independent of the biguanide. This constitutes the second active ingredient and may be formulated to provide an immediate release of the DPP4 inhibitor. In one embodiment of the present invention the DPP4 inhibitor is applied in the form of a layer to a controlled or slow released core comprising the a biguanide as a layer using a binder and other conventional pharmaceutical excipients such as absorption enhancers, surfactants, plasticizers, antifoaming agents and combinations of the foregoing. An absorption enhancer may be present in the DPP4 inhibitor layer in an amount up to about 30% w/w in comparison to the weight of the DPP4 inhibitor. A binding agent may be present in an amount up to 150% w/w of the DPP4 inhibitor. A second active ingredient immediate release formulation may be incorporated into a single dosage form by coating onto the membrane or slow release coating of the dosage form by conventional methods. Alternatively, it may also be incorporated by any pharmaceutically acceptable method into a single dosage form with the first active ingredient. The incorporation of the second active ingredient may be performed, among others, by commonly used processes selected from the group consisting of drug layering, lamination, dry compression, deposition and printing.

When the DPP4 inhibitor is coated onto a membrane or slow release coating of an osmotic tablet core, the DPP4 inhibitor coating should be applied from a coating solution or suspension that employs an aqueous solvent, an organic solvent or a mixture of an aqueous and an organic solvent. Typical organic solvents include acetone, isopropyl alcohol, methanol and ethanol. Whenever a mixture of aqueous and organic solvents is employed, the ratio of water to organic solvent should be in the range from 98:2 to 2:98, preferably 50:50 to 2:98, most preferably 30:70 to 20:80 and most preferably from about 25:75 to about 20:80. When a mixed solvent system is employed, the amount of binder required for coating the DPP4 inhibitor onto the membrane or a slow release coating may be reduced. For example, successful coatings have been obtained from a mixed solvent system where the ratio of binder to DPP4 inhibitor is 1:9 to 1:11. Although acceptable coatings can be obtained when the DPP4 inhibitor coat is applied directly to the membrane or slow release coating, a preferred approach is to first coat the membrane or slow release coating with a seal coat prior to the application of the DPP4 inhibitor coating. The DPP4 inhibitor coating solution or suspension may also contain a surfactant and a pore forming agent such as sodium chloride, potassium chloride, sucrose, sorbitol, mannitol, polyethylene glycols (PEG), propylene glycol, hydroxypropyl cellulose, hydroxypropyl methycellulose, hydroxypropyl methycellulose phthalate, cellulose acetate phthalate, polyvinyl alcohols, methacrylic acid copolymers, poloxamers. In an alternative embodiment, the pharmaceutical composition of the present invention may also comprise an effective immediate release amount of the biguanide. The effective immediate release amount of biguanide may be coated onto the membrane or slow release coating of the dosage form or it may be incorporated into the membrane or slow release coating.

In addition, various diluents, excipients, lubricants, dyes, pigments, dispersants, etc., which are disclosed in Remington's Pharmaceutical Sciences (1995), may be used to optimize the above listed formulations of the subject invention.

Biguanides, such as metformin are commonly administered in dosage forms containing 500 mg, 750 mg, 850 mg, and 1000 mg. DPP4 inhibitors, for example sitagliptin, is commonly administered in dosage forms containing 25 mg, 50 mg and 100 mg⁶. The present invention is intended to encompass the above listed therapeutic combinations, without providing a specific example of each possible combination of compounds and their respective dosage amounts.

A preferred embodiment of the pharmaceutical composition form, using Sitagliptin Phosphate as described in U.S. Pat. No. 6,303,661 will have the following composition:

TABLE 1

| | Range percent | Preferred Range % |
|---|---|---|
| First Active Ingredient | | |
| Drug | 50-98% | 75-95% |
| Binder | 0.1-40% | 3-15% |
| Absorption Enhancer | 0-20% | 2-10% |
| Lubricant | 0-5% | 0.5-1% |
| Coat | | |
| Polymer | 50-99% | 75-95% |
| Flux Enhancer | 0-40% | 2-20% |
| Plasticizer | 0-25% | 2-15% |
| Second Active | | |
| Ingredient | | |
| Drug | 0.1-20% | 1-10% |
| Binder | 0.1-20% | 1-15% |
| Surfactant | 0-20% | 0.1-15% |
| Pore Former | 0-25% | 0.1-15% |
| Polymer (Optional) | 0-30% | 0.1-20% |

The dosage forms prepared according to the present invention exhibit the following dissolution profile when tested in a USP Type 2 apparatus at 75 rpm in 900 ml of simulated intestinal fluid (pH 7.5 phosphate buffer) and at 37° C.:

TABLE 2
Dissolution Profile

| | | |
|---|---|---|
| Time hours | Percent Release | Preferred Range |

Biguanide

| | | |
|---|---|---|
| 2 | 0-25% | 0-15% |
| 4 | 10-45% | 20-40% |
| 8 | 30-90% | 45-90% |
| 12 | >50% | >60% |
| 16 | >60% | >60% |
| 20 | >70% | >70% |

DPP4 Inhibitor

| | | |
|---|---|---|
| 1 | >85% | >85% |

It has been discovered that the selection of the excipients for use in the DPP4 ingredient layer of the dosage form can greatly affect the release characteristics, potency and stability of the DPP4 inhibitor. Therefore, in an alternate embodiment of the present invention, the composition of the DPP4 inhibitor component of the present invention should be selected so that not less than 85%, preferably not less than 90% and most preferably not less than 95% of the DPP4 inhibitor is released from the dosage form within 120 minutes, preferably within 90 minutes and most preferably within 60 minutes when tested according to the United States Pharmacopeia (USP) 26, with Apparatus 1 at 100 rpm, 37′ C. and 900 ml of 0.3 M KCl-HCl Buffer, pH 2.0.

Further the excipients for use in the DPP4 inhibitor layer of the dosage form should be selected so that the total DPP4 inhibitor related compounds or impurities in the final dosage form are not more than 0.6%, preferably not more than 0.5% and most preferably not more than 0.25% and each individual DPP4 inhibitor related compound or impurity in the final dosage form is not more than 0.25%, preferably not more than 0.2% and most preferably not more than 0.1%. The DPP inhibitor related compounds or impurities in the final dosage form are determined by High Performance Liquid Chromatography (HPLC) using a YMC-ODS-AQ, 5.mu.m, 120 ANG., 4.6.times.250 mm or equivalent column, a 0.1 M ammonium acetate buffer:acetonitrile:glacial acetic acid (25:25:1) mobile phase, about a 40.mu.L injection volume, 0.7 mL/min flow rate, 25′C column temperature and 269 nm wavelength for the UV detector.

### PHARMACEUTICAL COMPOSITION

The following are provided by way of examples only and are in no means intended to be limiting.

### Example 1

The Table 3 shows the representative example of a pharmaceutical composition of a slow release comprising biguanide: Metformin HCl and a DPP inhibitor: Sitagliptin Phosphate

TABLE 3

| First Active Ingredient | Perecent of Core |
|---|---|
| Metformin HCl | 90.54% |
| Povidone K 301 USP | 4.38% |
| Sodium Tribasic Phosphate | 4.58% |
| Magnesium Stearate | 0.50% |
| Membrane | Percent of membrane |
| Cellulose Acetate (398-10)′ 85% | 85.00% |
| Triacetin 5% PEG 400 10% | |
| Triacetin | 5.00% |
| PEG 400 | 10.00% |
| Second Active Ingredient | Percent of second layer |
| Sitagliptin Phosphate | 43.50% |
| Tween | 2.00% |
| HPMC | 54.50% |

The slow-release tablet containing 850 mg of metformin HCl and 50 mg sitagliptin phosphate is prepared using a three step process: 1) Granulation, 2) Tabeting and 3) Membrane coating process. An optional Seal Coating may be done on the core tablet. These are described below:

1. Granulation

The Povidone, K-30, and sodium tribasic phosphate are dissolved in purified water. The metformin HCl is collected in a clean, polyethylene-lined container after it is delumped by passing it through a 40 mesh screen. The delumped metformin HCl is then added to a top-spray fluidized bed granulator and granulated by spraying the binding solution of Povidone and sodium tribasic phosphate at an inlet air temperature of 50-70′ C, an atomization air pressure of 1-3 bars and a spray rate of 10-100 ml/min. Once the binding solution is depleted, the granules are dried in the granulator until the loss on drying is less than 2% and are passed through a comil equipped with the equivalent of an 18 mesh screen.

2. Tableting

The magnesium stearate and metformin HCl are thoroughly blended together, after passing magnesium stearate through a 40 mesh stainless steel screen, for approximately five (5) minutes. Following this, the granules are compressed on a rotary press fitted with (fraction ( 15/32)″ round standard concave punches. As stated, the orifice may be formed by any means commonly employed in the pharmaceutical industry.

2a. Seal Coating (Optional)

Optionally the seal coating of the tablet can by first dissolving the Opadry material, preferably Opadry Clear, in purified water and spraying the Opadry solution onto the core tablet using a pan coater at an exhaust air temperature of 38-42′ C degree, an atomization pressure of 28-40 psi and a spay rate of 10-15 ml/min. The core tablet is coated with the sealing solution until a theoretical coating level of approximately 2-4% is obtained.

3. Membrane Coating Process

A homogenizer was used for dissolving the cellulose acetate is dissolved in acetone. The polyethylene glycol 400 and triacetin are added to the cellulose acetate solution and stirred until a clear solution is obtained. The clear membrane coating solution is then sprayed onto the seal coated tablets using a fluidized bed coater employing the following conditions: product temperature of 16-22.degree. C.; atomization pressure of approximately 3 bars and spray rate of 120-150 ml/min. The sealed core tablet is coated until a theoretical coating level of approximately 3% is obtained. Tween 80 and hydroxypropyl methylcellulose are dissolved in purified water. Sitagliptin Phosphate is then dispersed into this solution. The resulting suspension is then sprayed onto the above-membrane-coated tablets.

### Example 2

The Table 4 shows the representative example of a pharmaceutical composition of a slow release comprising biguanide and a DPP inhibitor using Sodium Lauryl Sulfate.

TABLE 4

| First Active Ingredient | Percent composition of core |
|---|---|
| Metformin HCl | 88.55% |
| Povidone K 301 USP | 6.38% |
| Sodium Lauryl Sulfate | 4.57% |
| Magnesium Stearate | 0.50% |
| Membrane | Percent of membrane |
| Cellulose Acetate (398-10)′ 85% | 85.00% |
| Triacetin 5% PEG 400 10% | |
| Triacetin | 5.00% |
| PEG 400 | 10.00% |
| Second Active Ingredient | Percent of second layer |
| Sitagliptin Phosphate | 43.50% |
| Tween | 2.00% |
| HPMC | 54.50% |

The slow-release tablet containing 850 mg of metformin HCl and 50 mg sitagliptin phosphate using a different excipient Sodium Lauryl Sulfate is prepared using a three step process as described above in Example 1 except the grannulation process was modified as below.

1. Granulation

The metformin HCl and sodium lauryl sulfate are delumped by passing them through a 40 mesh screen and collecting them in a clean, polyethylene-lined container. The povidone, K-90, is dissolved in purified water. The delumped metformin HCl and sodium lauryl sulfate are then added to a top-spray fluidized bed granulator and granulated by spraying with the binding solution of povidone under the conditions of an inlet air temperature of 50-70′ C, a atomization air pressure of 1-3 bars and a spray rate of 10-100 ml/min. Once the binding solution is depleted, the granules are dried in the granulator until the loss on drying is less than 2%. The dried granules are passed through a comil equipped with the equivalent of an 18 mesh screen.

The rest of the steps in the manufacturing process: Tableting, Optional Seal Coating and Membrane coating, were as described in Example 1

### Example 3

The Table 5 shows the representative example of a pharmaceutical composition of a slow release comprising biguanide, for Example 500 mg of Metformin HCl and 50 MG of Sitagliptin Phosphate;

TABLE 5

| | Amount mg/tablet |
|---|---|
| First Active Ingredient | |
| Metformin HCl | 500.0 |
| Povidone K 301 USP | 36.0 |
| Sodium Lauryl Sulfate | 25.8 |
| Magnesium Stearate | 2.8 |
| Seal Coat | |
| Opadry Clear (YS 1-7006) | 23.5 |
| Semi permeable coat | |
| Cellulose Acetate (398-10) | |
| NF | 23.6 |
| Triacetin | 1.4 |
| PEG 400 | 2.8 |
| Second Active Ingredient | |
| Sitagliptin Phosphate | 50.0 |
| Tween | 2.0 |
| Polyplasdone XL | 15.0 |
| Opadry Clear (YS 1-7006) | 8.5 |

The manufacturing process for a slow release tablet containing 500 mg of metformin HCl and 50 mg sitagliptin phosphate is described below:

I. First Active Ingredient: A 500 mg metformin membrane coated tablet is prepared as described in Example 2 above except that compound cup toolings are used during tableting.

II. Second Active Ingredient Layering: An immediate release amount of sitagliptin phosphate is applied to the 500 mg metformin HCl membrane coated tablet prepared in step I.

The sitagliptin coating is directly applied to the 500 mg metformin HCl membrane coated tablets. The sitagliptin coating is prepared by dissolving 0.252 kg of Opadry Clear, 0.269 kg of Polyplasdone XL and 0.036 kg of Tween 80 in 9.908 kg of purified water using a homogenizer.

Once these ingredients are dissolved, 0.296 kg of sitagliptin phosphate is dispersed into the solution and homogenized. The homogenized dispersion is then directly applied to the 500 mg metformin HCl membrane coated tablets using a 24″ O'Hara Labcoat III pan coater. The experimental conditions are at a Spray Rate 15-27 mL/min, an Exhaust Temperature 42-47′ C, an Atomization Air Pressure 25 psi, Pan Speed 5-9 rpm and at an Inlet Air Flow 300-400 CFM

Once the Sitagliptin coating has been applied to the 500 mg metformin-HCl membrane coated tablet, an aesthetic or color coating of Opadry white is applied to the sitagliptin coated tablet. The color coating is prepared by dispersing 0.179 kg of Opadry White in 1.791 kg of purified water. The Opadry White suspension is applied to the sitagliptin coated tablet using a 24″ O'Hara Labcoat III pan coater. The experimental conditions were at a Spray Rate 20-35 mL/min, an Exhaust Temperature 35-45′ C, an Atomization Air Pressure 25 psi, a Pan Speed 9 rpm and an Inlet Air Flow 390-500 CFM. Once the color coating is applied, the tablets are polished using 0.036 kg of Candelilla wax powder. In addition, Opadry White and Cindrella Wax Powder were used in 10 mg and 2 mg respectively per tablet

### Example 4

The Table 6 shows the representative example of a pharmaceutical composition of a slow release comprising biguanide, for Example 500 mg of Metformin HCl and 50 MG of Sitagliptin Phosphate.

TABLE 6

| | Amount mg |
|---|---|
| First Active Ingredient | |
| Metformin HCl | 500.0 |
| Povidone K 301 USP | 36.0 |
| Sodium Lauryl Sulfate | 25.8 |
| Magnesium Stearate | 2.8 |
| Seal Coat | |
| Opadry Clear (YS 1-7006) | 23.5 |
| Semi permeable coat | |
| Cellulose Acetate (398-10) | |
| NF | 23.6 |
| Triacetin USP | 1.4 |
| PEG 400 | 2.8 |
| Seal coat | |
| Opadry Clear (YS 1-7006) | 13.8 |
| Second active ingredient | |
| Sitagliptin Phosphate | 50.0 |
| Tween 80 | 2.0 |
| Sodium Chloride | 4.3 |
| Opadry Clear (YS 1-7006) | 2.0 |

The manufacturing process for a slow release tablet containing 500 mg of metformin HCl and 50 mg sitagliptin phosphate is described below:

I. First Active Ingredient: A 500 mg metformin membrane coated tablet is prepared as described in Example 2 above except that compound cup toolings are used during tableting.

II. Second Active Drug Layering: An immediate release amount of sitagliptin phosphate is applied to the 500 mg metformin HCl seal coated tablet prepared in Step 1.

Seal Coating: The seal coating solution is prepared by dissolving 0.258 kg of Opadry Clear in 2.576 kg of purified water and spraying the solution onto approximately 12.088 kg of the 500 mg membrane coated metformin HCl tablet cores using a 24″ O'Hara Labcoat III pan coater. The seal coat is applied under the experimental conditions of a Spray Rate 20-35 mL/min, an Exhaust Temperature 35-45′ C, an Atomization Air Pressure 25 psi, a Pan Speed 9 rpm and at an Inlet Air Flow 390-500 CFM

The sitagliptin coating is applied to the seal coated 500 mg metformin HCl membrane coated tablets. The sitagliptin coating is prepared by dissolving 0.040 kg of Opadry Clear, 0.085 kg of sodium chloride and 0.040 kg of Tween 80 in 4.915 kg of purified water using a homogenizer. Once these ingredients are dissolved, 0.328 kg of sitagliptin phosphate is dispersed into the solution and homogenized. The homogenized dispersion is then applied to the seal coated 500 mg metformin HCl membrane coated tablets using a 24″ O'Hara Labcoat III pan coater. The experimental conditions for Sitagliptin coating were done at a Spray Rate 10-30 mL/gun/min, an Exhaust Temperature 35-45′ C, an Atomization Air Pressure 20-40 psi, a Pattern Air Pressure 20-40 psi, a Pan Speed 8-12 rpm, and at an Inlet Air Flow 250-450 CFM.

Once the sitagliptin coating has been applied to the seal coated 500 mg metformin HCl membrane coated tablets, an aesthetic or color coating of Opadry White is applied to the sitagliptin coated tablet. The color coating is prepared by dispersing 0.159 kg of Opadry White in 1.585 kg of purified water. The Opadry White suspension is applied to the sitagliptin coated tablet using conditions similar to those described above for application of the seal coating. Once the color coating is applied, the tablets are polished using 0.004 kg of Candelilla wax powder.

### Example 5

The Table 7 shows the representative example of a pharmaceutical composition of a slow release comprising biguanide, for Example 1000 mg of Metformin HCl and 100 MG of Sitagliptin Phosphate.

TABLE 7

| | Amount mg |
|---|---|
| First Active Ingredient | |
| Metformin HCl | 1000.0 |
| Povidone K 301 USP | 78.0 |
| Sodium Lauryl Sulfate | 51.7 |
| Magnesium Stearate | 5.7 |
| Seal Coat | |
| Opadry Clear (YS 1-7006) | 47.1 |
| Semi permeable coat | |
| Cellulose Acetate (398-10) | |
| NF | 15.8 |
| Triacetin | 0.9 |
| PEG 400 | 1.9 |
| Seal coat | |
| Opadry Clear (YS 1-7006) | 16.0 |
| Second Active Ingredient | |
| Sitagliptin Phosphate | 100.0 |
| Sodium chloride | 4.3 |
| Opadry Clear (YS 1-7006) | 3.0 |

The manufacturing process for a slow release tablet containing 1000 mg of metformin HCl and 100 mg sitagliptin phosphate is described below:

I. First Active Drug: A 1000 mg metformin membrane coated tablet is prepared as described in Example 2 above.

II. Second Active Drug: An immediate release amount of sitagliptin is applied to the 1000 mg metformin HCl membrane coated tablets prepared in step I.

The seal coating is prepared by dispersing 0.174 kg of Opadry. Clear in 3.478 kg of ethanol and mixing the dispersion for 15 minutes. The dispersion is than sprayed onto approximately 13.174 kg of the 1000 mg metformin HCl membrane coated tablets using a 24″ O'Hara Labcoat III pan is coater. The seal coat is applied to the 1000 mg metformin HCl membrane coated tablets under conditions of a Spray Rate 10-30 ml/gun/min, an Exhaust Temperature 25-45′ C, an Atomization Air Pressure of 20-40 psi, a Pan Speed between 6-12 rpms, a Pattern Air Pressure of 20-40 psi and an Inlet Air Flow of 250-450 CFM

The sitagliptin coating then is applied to the seal coated 1000 mg metformin HCl membrane coated tablets. The sitagliptin coating is prepared by dissolving 0.036 kg of Opadry Clear and 0.046 kg of sodium chloride in 5.344 kg of ethanol using a homogenizer. Once the ingredients are dispersed, 0.359 kg of sitagliptin is dispersed into the solution and homogenized. The homogenized dispersion is then applied to the seal coated 1000 mg metformin HCl membrane coated tablets using a 24″ O'Hara Labcoat III pan coater under experimental conditions of a Spray Rate between 10 to 30 mL/gun/min, an Exhaust Temperature of 25-45′ C, an Atomization Air Pressure of 20-40 psi, a Pan Speed between 6 to 12 rpm, a Pattern Air Pressure between 20-40 psi and an Inlet Air Flow of 250-450 CFM

Once the sitagliptin coating has been applied, an aesthetic or color coating of Opadry II White is applied to the sitagliptin coated tablets. The color coating is prepared by dispersing 0.220 kg of Opadry II White in 4.407 kg of ethanol. The Opadry II White suspension is than applied to the sitagliptin phosphate coated tablets using a 24″ O'Hara Labcoat III pan coater using conditions similar to those described above for the seal coating. Once the color coating is applied, the tablets are polished using 0.004 kg of Candelilla wax powder.

### Example 6

The Table 8 shows the representative example of a pharmaceutical composition of a slow release comprising biguanide, for Example 1000 mg of Metformin HCl and 100 MG of Sitagliptin Phosphate.

TABLE 8

| | Amount mg |
|---|---|
| First Active Ingredient | |
| Metformin HCl | 1000.0 |
| Povidone K 301 USP | 78.0 |
| Sodium Lauryl Sulfate | 51.7 |
| Magnesium Stearate | 5.7 |
| Seal Coat | |
| Opadry Clear (YS 1-7006) | 47.1 |
| Semi permeable coat | |
| Cellulose Acetate (398-10) | |
| NF | 15.8 |
| Triacetin | 0.9 |
| PEG 400 | 1.9 |
| Seal coat | |
| Opadry Clear (YS 1-7006) | 21.0 |
| Second Active Ingredient | |
| Sitagliptin Phosphate | 100.0 |
| Sodium chloride | 5.0 |
| Opadry Clear (YS 1-7006) | 3.7 |

The manufacturing process for a slow release tablet containing 1000 mg of metformin HCl and 100 mg sitagliptin phosphate is described below:

I. First Active Drug: A 1000 mg membrane coated tablet is prepared as described in Example 3 above.

II. Second Active Drug: An immediate release amount of sitagliptin phosphate is applied to the 1000 mg metformin HCl membrane coated tablets prepared in step I.

The seal coat is applied to the 1000 mg metformin HCl membrane coated tablet. The seal coating is prepared by dispersing 0.229 kg of Opadry Clear in 4.573 kg of alcohol USP and mixing the dispersion for 15 minutes. The dispersion is then sprayed onto approximately 13.68 kg of the 1000 mg metformin HCl tablet cores using a 24″ O'Hara Labcoat III pan coater with the nozzle tip set 4+/−2″ from the top of the static bed under the conditions of a Spray Rate of 25+/−10 mL/gun/min, an Exhaust Temperature of 25′ C+/−5′ C, an Atomization Air Pressure of 10-40 psi, a Pan Speed of 4-9 rpm, a Supply Air Flow of 200+/−100 CFM and a Pattern Air Pressure of 10-40 psi. The seal coating dispersion is continuously stirred until it is consumed during the coating process.

The sitagliptin coating then is applied to the seal coated 1000 mg metformin HCl membrane coated tablets. The sitagliptin coating is prepared by mixing 4.434 kg of alcohol USP and 1.250 kg of purified water (approximately a 78:22 alcohol to purified water ratio) and slowly dispersing 0.040 kg of Opadry Clear into the solvent mixture. Once the Opadry Clear is dispersed, it is homogenized for about 10 minutes. Once the Opadry Clear dispersion is homogenized, 0.054 kg of sodium chloride is added to the dispersion and homogenized for about 2 minutes. After the sodium chloride is homogenized, 0.360 kg of sitagliptin phosphate is slowly dispersed into the solvent mix and then homogenized for about 10 minutes. Once the sitagliptin phosphate is homogenized, the homogenizer is removed from the mixing vessel and replaced with an air mixer and mixed for an additional 15 minutes. The sitagliptin suspension is stirred until the suspension is consumed during the coating process. The sitagliptin suspension is applied to the seal coated 1000 mg metformin HCl membrane coated tablet cores using a 24″ O'Hara Labcoat III pan coater with the nozzle tip set 4+/−2″ above the top of the static bed and carried at a Spray Rate 25+/−10 mL/gun/min, a Exhaust Temperature 25′C+/−5′ C, a Atomization Air Pressure 10-40 psi, a Pan Speed 4-9 rams, a Pattern Air Pressure 10-40 psi and a Supply Air Flow 200+/−100 CFM

Once the sitagliptin coating has been applied to the seal coated 1000 mg metformin HCl membrane coated tablets, an aesthetic coating of Opadry II White is applied to the sitagliptin coated tablet. The aesthetic coating is prepared by dispersing 0.235 kg of Opadry II White (Y-22-7719) in 4.691 kg of alcohol USP and mixing the dispersion for about 1 hour. The Opadry II White dispersion is than sprayed onto the sitagliptin phosphate coated tablets using a 24″ O'Hara Labcoat III pan coater with the nozzle tip set 4.+−0.2″ from the top of the static bed and the process carried at a Spray Rate 25+/−10 mL/gun/min, an Exhaust Temperature 25′C+/−5′C, a Atomization Air Pressure 10-40 psi, a Pan Speed 4-9 rpm, a Supply Air Flow 200+/−100 CFM and a Pattern Air Pressure 10-40 psi. The color coating dispersion is continuously stirred until the dispersion is consumed during the coating process.

Once the aesthetic coating suspension is consumed, the tablets are dried in the coating pan for about 5 minutes with a pan speed of about 2-8 rpms and an exhaust temperature of 25′C+/−5′ C. Once the tables are dried, the exhaust air is turned off and the pan speed is adjusted to about 3-4 rpms and 0.004 kg of Candellia wax powder that had been passed through a 60 mesh screen is sprinkled onto the tablets have rolled in the wax for about 5 minutes the exhaust air is turned on and the tables are rolled for an additional 10 minutes.

The finished polished tablet exhibited the following sitagliptin phosphate dissolution profile (Table 9) when tested in USP apparatus type I at 100 rpm in a pH 2.0 HCl-0.3M KCl buffer solution: More than 85% of sitagliptin is dissolved with 15 minutes

TABLE 9

| Time | | Percent released |
|---|---|---|
| 5 | | 60.00 |
| 10 | | 75.00 |
| 15 | | 85.00 |
| 45 | | 95.00 |
| 90 | min | 95.00 |
| 120 | min | 95.00 |

### Example 7

The table 10 shows the representative example of a pharmaceutical composition of a slow release comprising biguanide: Metformin HCl and yet another DPP inhibitor: Vildagliptin

TABLE 10

| First Active Ingredient | Perecent of Core |
|---|---|
| Metformin HCl | 90.54% |
| Povidone K 301 USP | 4.38% |
| Sodium Tribasic Phosphate | 4.58% |
| Magnesium Stearate | 0.50% |
| Membrane | Percent of membrane |
| Cellulose Acetate (398-10)′ 85% | 85.00% |
| Triacetin 5% PEG 400 10% | |
| Triacetin | 5.00% |
| PEG 400 | 10.00% |
| Second Active Ingredient | Percent of second layer |
| Vildagiptin | 43.50% |
| Tween | 2.00% |
| HPMC | 54.50% |

A preferred embodiment of the pharmaceutical composition form, using Vildagliptin as described in WO 9819998, WO 0034241, U.S. Pat. No. 6,110,949 that are incorporated herein as reference, will have the following composition: as in Table 10

TABLE 11

| First Active Ingredient | Percent composition of core |
|---|---|
| Metformin HCl | 88.55% |
| Povidone K 301 USP | 6.38% |
| Sodium Lauryl Sulfate | 4.57% |
| Magnesium Stearate | 0.50% |
| Membrane | Percent of membrane |
| Cellulose Acetate (398-10)′ 85% | 85.00% |
| Triacetin 5% PEG 400 10% | |
| Triacetin | 5.00% |
| PEG 400 | 10.00% |
| Second Active Ingredient | Percent of second layer |
| Vildagliptin | 43.50% |
| Tween | 2.00% |
| HPMC | 54.50% |

The manufacturing of a pharmaceutical composition comprising a slow release Metformin HCl and Vildagliptin can be carried using disclosed in Examples 1-6

While certain preferred and alternative embodiments of the invention have been set forth for purposes of disclosing the invention, modifications to the disclosed embodiments may occur to those who are skilled in the art. Accordingly, the appended claims are intended to cover pharmaceutical compositions comprising a slow release biguanide with all DPP4 inhibitors, all embodiments of the invention and modifications thereof which do not depart from the spirit and scope of the invention

### Clinical Studies

Study 1: A total of 11 subjects were enrolled in the study and all of them randomly received drugs as follows:
1. Example 6 (Combination Drug of Slow Release 1000 mg Metformin+100 mg Sitagliptin)
2. Reference B (Januvia 100 mg+2 Tablets of Glucophage XR 500 mg)

Each study included two treatment phases wherein each phase was separated by washout period of 21 days. Subjects were randomized to receive one of the above two regimens as randomly assigned by Latin Square and each subject crossed to each regimen according to the randomization sequence until all subjects have received all two regimens (with twenty one week separating each regimen). Blood samples were centrifuged within 2 hours of collection and the plasma were separated and frozen at −10′ C or lower until assayed. HPLC Analysis was carried out using stand techniques known to the person skilled in art using sitagliptin phosphate and internal standard (NC-34) were used.

### REFERENCES

1. Vidon, N., Chaussade, S., Noel, M., Franchisseur, C., Huchet, B., Bernier, J. J. (1988), Diabetes Research and Clinical Practice, 4, 223-239.
2. Recent Patents on Endocrine, Metabolic & Immune Drug Discovery 2007, Vol. 1, No. 1.
3. Pentikainen, P. J. (1986), International Journal of Clinical Pharmacology, Therapy and Toxicology, 24, 213-220.
4. Noel, D. S. (1980), Kinetic study of normal and sustained release dosage forms of metformin in normal subjects, Journal of International Biomedical Information and Data, 1980, pp. 9 to 20.
5. Karttunen et al (1983), The pharmacokinetics of metformin: a comparison of the properties of a rapid-release and a sustained-release preparation, Int. J. Clin. Pharmacology, Therapy and Toxicology, Vol. 21, No. 1, pp. 31-36.
6. FDA labeling information [online], (2006).
7. Raz, I. et al. Efficacy and safety of the dipeptidyl peptidase-4 inhibitor sitagliptin as monotherapy in patients with type 2 diabetes mellitus. Diabetologia 49, 2564-2571 (2006).
8. Aschner, P. et al. Effect of the dipeptidyl peptidase-4 inhibitor sitagliptin as monotherapy on glycemic control in patients with type 2 diabetes. Diabetes Care 29, 2632-2637 (2006).
9. Charbonnel, B. et al. Efficacy and safety of the dipeptidyl peptidase-4 inhibitor sitagliptin added to ongoing metformin therapy in patients with type 2 diabetes inadequately controlled with metformin alone. Diabetes Care 29, 2638-2643 (2006).
10. Rosenstock, J. et al. Efficacy and safety of the dipeptidyl peptidase-4 inhibitor sitagliptin added to ongoing pioglitazone therapy in patients with type 2 diabetes: a 24-week, multicenter, randomized, double-blind, placebo-controlled, parallel-group study. Clin. Ther. 28, 1556-1568 (2006).
11. U.S. Pat. No. 3,845,770,
12. U.S. Pat. No. 3,916,899,
13. U.S. Pat. No. 4,008,719,
14. U.S. Pat. No. 4,034,758,
15. U.S. Pat. No. 4,036,228,
16. U.S. Pat. No. 4,522,625,
17. U.S. Pat. No. 4,612,008
18. U.S. Pat. No. 4,077,407,
19. U.S. Pat. No. 4,783,337,
20. U.S. Pat. No. 5,071,607,
21. U.S. Pat. No. 6,011,049,
22. U.S. Pat. No. 6,099,859,
23. U.S. Pat. No. 6,110,949
24. U.S. Pat. No. 6,153,632,
25. U.S. Pat. No. 6,340,475,
26. U.S. Pat. No. 6,403,121
27. U.S. Pat. No. 6,475,521
28. U.S. Pat. No. 6,451,342,
29. U.S. Pat. No. 6,524,621
30. U.S. Pat. No. 6,676,966
31. WO 01/3594,
32. WO 01/3594,
33. U.S. Pat. No. 7,125,873,
34. WO 9819998,
35. WO 0034241,

We claim:

1. A pharmaceutical composition comprising a slow release biguanide and a DPP4 inhibitor wherein the said composition comprising: a) Slow release core comprising a biguanide and at least one pharmaceutically acceptable excipient, b) An immediate release coat comprising a DPP4 inhibitor

2. A pharmaceutical composition of claim 1 wherein the DPP4 inhibitor is an entity that inhibits dipeptidyl peptidase IV protease.

3. A pharmaceutical composition of claim 1 wherein the biguanide is selected from comprising metformin, phenformin and buformin or their pharmaceutically equivalent salts or derivatives

4. A pharmaceutical composition of claim 1 wherein the DPP4 inhibitor is selected from a group comprising Sitagliptin, Vildagliptin, Saxagliptin, SYR 522, PHX1149, GRC-8200 and SSR-162369

5. A method of administering a pharmaceutical composition comprising a slow release biguanide and a DPP4 inhibitor wherein the said composition comprising: a) Slow release core comprising a biguanide and at least one pharmaceutically acceptable excipient, b) An immediate release coat comprising a DPP4 inhibitor

6. A method of administration of claim 5, wherein the biguanide is selected from comprising metformin, phenformin and buformin or their pharmaceutically equivalent salts or derivatives

7. A method of administration of claim 5, wherein the DPP4 inhibitor is an entity that inhibits dipeptidyl peptidase IV protease.

8. A method of administration of claim 7, wherein the DPP4 inhibitor is selected from a group comprising Sitagliptin, Vildagliptin, Saxagliptin, SYR 522, PHX1149, GRC-8200 and SSR-162369

9. A pharmaceutical composition of claim 1 wherein at least 95% of the DPP4 inhibitor is released within 120 minutes

10. A pharmaceutical composition according to claim 1 wherein the excipient is selected from a group comprising an adjuvant, a preservative, an antioxidant, a thickening agent, a chelating agent, an antifungal agent, an antibacterial agent, an isotonic agent, a flavoring agent, a sweetening agent, an anti-foaming agent, a colorant, a diluent, a moistening agent, a parietal cell activator, or a combination of thereof
